(19) United States
(12) Patent Application Publication
(10) Pub. No.: US 2003/0192536 A1
(43) Pub. Date: Oct. 16, 2003

(54) Personal containment system with isolated blower

(76) Inventors: Raymond Odell, Hertfordshire (GB); Jason A. Graves, Berkshire (GB); Martin J. Avery, Middlesex (GB)

Correspondence Address: 3M INNOVATIVE PROPERTIES COMPANY, PO BOX 33427, ST. PAUL, MN, 55133-3427, US

(21) Appl. No.: 10/121,306

(22) Filed: Apr. 12, 2002

(51) Int. Cl. A62B 18/00; A61M 15/00

(52) U.S. Cl. 128/201.22; 128/202.19; 128/204.18

(57) ABSTRACT

A personal containment system is disclosed. The personal containment system includes a generally fluid-tight barrier having an inner surface and an outer surface. The generally fluid tight barrier is adapted to at least partially define an inner environment interfacing with the inner surface, and an outer environment interfacing with the outer surface. The personal containment system also includes an air delivery system powered by a blower connected through the generally fluid-tight barrier. The blower is generally isolated from the outer environment and draws purified air from a filter in fluid communication with the blower that is located outside of the containment. Air from the outer environment is passed into the inner environment by the blower through a filter and exhausted through a one-way valve or a exhaust filter.

### BACKGROUND

The present disclosure relates to a personal containment system, such as a protective suit adapted to isolate a person from a contaminated environment. More particularly, the present disclosure relates to a personal containment system with an air delivery system.

Personal containment systems are useful to protect a user from a variety of harmful chemical or biological agents. Many examples of personal containment systems are known, such as gas-masks, protective suits, protective tents, casualty bags for injured persons, and the like. These systems isolate a body from contaminants. Often, the contaminants include respiratory hazards, and the systems must employ respirators or air delivery systems so that the user is able to breathe when isolated from the environment. A variety of respirators are known and described below.

Certain gas masks and other systems employ a non-powered purifying respirator. Air is drawn into the system through a filter by the user's breathing action. When the wearer draws a breath, negative pressure is created in the mask and air is drawn in through the filter. When the user expels a breath, spent air leaves the system through a valve.

Powered air-purifying respirators, or PAPRs are employed to continually supply positive pressure in a personal containment system. A PAPR includes a filter attached to a blower that provides filter air to into the personal containment system. Often a conduit is attached to the blower, and the conduit is attached to the hood or a spigot on a protective garment. The PAPR may be powered by a battery, pneumatically, or with an external power source. When used with a protective garment, the PAPR is typically worn on a belt that is attached around a user's waist. PAPRs are generally used in industrial applications where the environmental hazards are well defined and quantified.

A self-contained breathing apparatus (SCBA) is another variety of respirator used in a personal containment system. A SCBA system typically employs a source of portable oxygen, and a breathing device that supplies the oxygen to a user. Often the SCBA system is worn under the protective garment. SCBA systems are often used when the nature of the hazard is not known. For example, the contaminated environment could be void of oxygen. In many applications, the user of an SCBA system will only remain in the contaminated environment for up to a half hour. The user wearing the SCBA will assess the nature of the contaminant and remove any injured persons requiring medical assistance.

These personal containment systems include both advantages and disadvantages. Often, users will prefer a continuous supply of oxygen such as from a PAPR or a SCBA system to a non-powered supply. A SCB system permits the user to enter into a greater number of environments than other systems. And a PAPR system is less expensive to use and maintain than a SCBA system. However, a PAPR system is prone to contamination in that it is exposed to the environment. Contamination of the PAPR is especially problematic in that to remove the contaminant requires cleaning of the many exposed components of the blower and associated parts. The blower is prone to deteriorate more quickly when exposed to corrosive conditions, or otherwise, in both the contaminated environment and in cleaning. In addition, the blower is often driven by an electric power supply and uses an electric motor. Electric sparks or the like do not interact well with all environments

### SUMMARY

The present invention is directed to a system that addresses the disadvantages of current PAPR systems yet is less expensive to use and maintain than a SCBA system. The present invention is directed to a personal containment system that includes a generally fluid-tight barrier having an inner surface and an outer surface. The generally fluid tight barrier is adapted to at least partially define an inner environment interfacing with the inner surface, and an outer environment interfacing with the outer surface. The personal containment system also includes an air delivery system powered by a blower connected through the generally fluid-tight barrier. The blower is generally isolated from the outer environment and draws purified air from a filter in fluid communication with the blower that is located outside or on the surface of the containment. Air from the outer environment is passed into the inner environment by the blower through a filter and exhausted through a one-way valve or a exhaust filter. In another aspect of the invention, the filter and blower are attached together with an interface that can provide a mechanical connection and seal the barrier.

The personal containment system has several advantages. For example, the personal containment system includes an isolated blower that does not need decontamination after use, thus saving expense. The personal containment system is suitable for use in situation where a contaminated environment is known to include oxygen, but otherwise the hazard is generally unknown. Further, the isolated blower enables the use of the personal containment system in environments where electric sparks or the like can provide a hazard. Accordingly, the personal containment system is adapted for use in more environments than typical PAPR systems. Still further, the personal containment system is also much less expensive and easier to maintain than a SCBA system.

### BRIEF DESCRIPTION OF THE DRAWINGS

FIG. 1 shows a schematic view of a personal containment system constructed in accordance with the present invention.

FIGS. 2-6 show schematic views of various examples of personal containment systems indicated in FIG. 1.

FIG. 7 shows a perspective view of a personal containment system, indicated in FIG. 1, in the form of a chemical suit.

FIG. 8 shows an exploded perspective view of a portion of the personal containment system of FIG. 7.

FIG. 9 shows an exploded view of a portion of the personal containment system indicated in FIG. 4.

### DETAILED DESCRIPTION

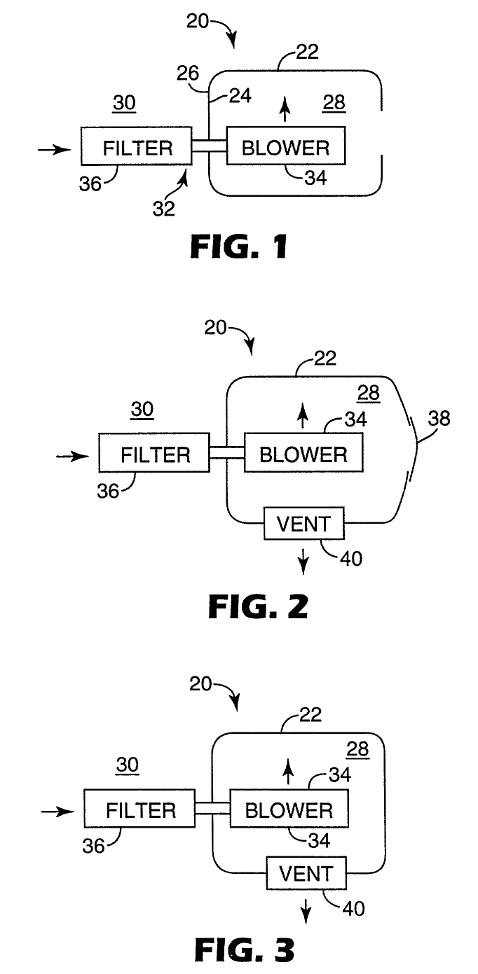

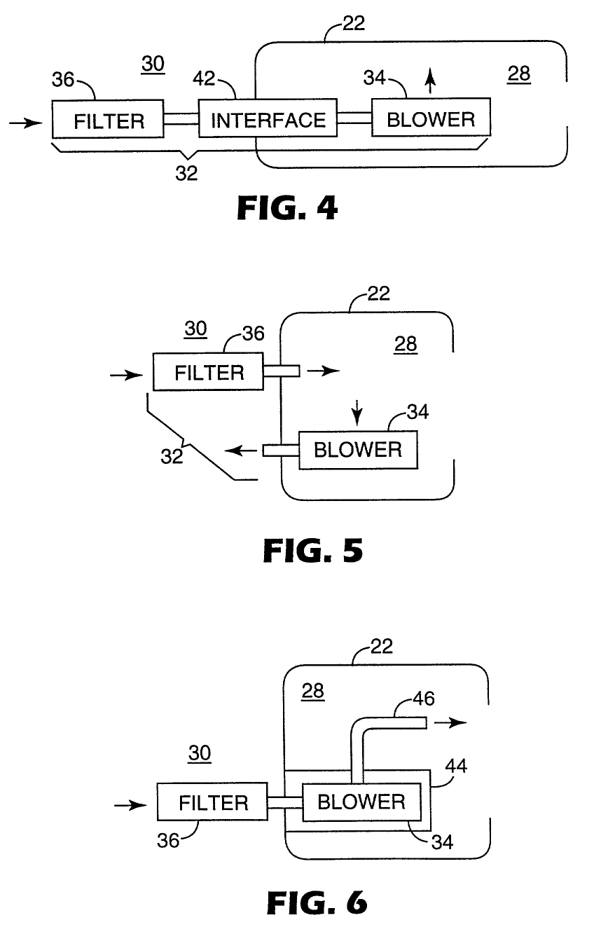

FIG. 1 shows a schematic view of an example, or embodiment, of the personal containment system 20 constructed in accordance with the present disclosure. The personal containment system 20 includes a generally fluid-tight barrier 22 having an inner surface 24 and an outer surface 26. The generally fluid tight barrier 22 adapted to at least partially define an inner environment 28 interfacing with the inner surface 24, and an outer environment 30 interfacing with the outer surface 26. The personal containment system 20 also includes an air delivery system 32 powered by a blower 34 connected through the generally fluid-tight barrier 22. The blower 34 is generally isolated from the outer environment 30 and draws filtered air from a filter 36 in fluid communication with the blower 34, and the filter is located in the outer environment 30. Air from the outer environment 30 is passed into the inner environment 28 by the blower 34 from the filter 34. FIGS. 2-5 show alternative embodiments of the general personal containment system 20 of FIG. 1, where like parts have like reference numerals.

The personal containment system 20 can be one of a variety of protection systems that may be suitable for protecting living things from a contaminated or hazardous environment. For example, the personal containment system can be a protective garment such as commonly known chemical suits, hoods or masks. Other examples include a tent or a casualty bag. Still other examples, both known and unknown, are intended to fall within the scope of the disclosure. The personal containment system at least partially defines an inner environment 28. The inner environment 28 is intended to be habitable and contaminant-free when worn in a contaminated or hazardous outer environment 30. The personal containment system is described as at partially defining an inner environment because, in the case of a suit, the suit may have openings for at least the limbs and head. In such a case, the suit may be worn with gloves, boots and a hood, for example. In another embodiment, one or more of the gloves boots and hood may be integral to the suit. In still another embodiment, the barrier may include the attachments such as one or more of the gloves, boots, and hood, which are not integral to the suit, but are connected to it in a known manner to protect a user. In cases where the personal containment system is a hood, there exists an opening at the neck, an so on.

FIG. 2 is a schematic view of the personal containment system 20 including an attachment 38 to define the inner environment 28. The attachment 38 can include one or more pieces. Accordingly, FIG. 2 can be a schematic representation of a suit with attached gloves, boots and hood, or the like. FIG. 3 is a schematic representation of a personal containment system 20 which is either self sealing or all attachments are integral with the generally fluid tight barrier 22. FIGS. 2 and 3 also include a vent 40 to permit gasses to escape. In one embodiment, the vent 40 is a one-way valve that opens automatically after the pressure within the inner environment 28 has reached a certain threshold. More than one vent 40 can be used. The vent 40 can be attached to the barrier 22, the attachment 38, or both. In another embodiment, the vent is an exhaust filter.

The generally fluid tight barrier 22 is typically over 90 percent fluid tight, depending on the application. In one example, the barrier 22 is suited for liquid applications, which typically means a mist or jet of liquid can be incident on the barrier 22 and the barrier will be impervious to the liquid. For example, a barrier that is impervious to liquid may be only 95 percent gas tight. In another application, the barrier may be constructed from a material that is impervious to gas. Often, any seams in the material are taped or welded to also be fluid tight. Accordingly, the generally fluid tight barrier is generally impervious to the contaminant of a particular application, and does not necessarily hermetically seal the inner environment 28 from the outer environment 30.

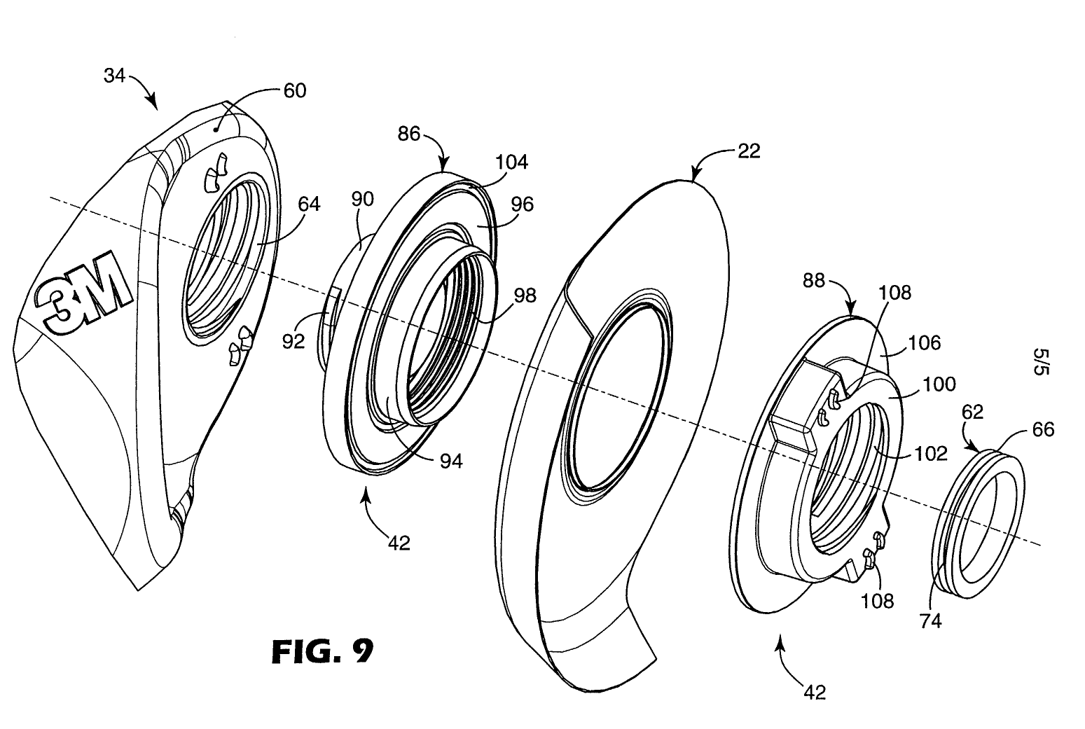

FIGS. 1, 4 and 5 shows schematic examples of how the air delivery system 32 can be connected through the barrier 22. In FIG. 1, the filter 36 is connected directly to the blower 34 through the barrier 22. In this embodiment, the air delivery system 32 is simply a commercially available system with no intermediate attachments between the filter 36 and the blower 34. In FIG. 4, the filter 36 is connected to the blower 34 with an interface 42. The filter 36 is attached to the interface 42 that is attached to the blower 34. One example of an interface 42 is described below in relation to FIG. 9. The air delivery system 32 can also include a conduit, indicated in FIG. 6 at 46, that delivers air into the inner environment 28. In FIGS. 1 and 4 the blower 34 draws air directly through the filter 36. The filter 36 is in fluid communication with the blower 34 in that a mechanical connection is established between the two. Air from the outer environment 30 is drawn into the air delivery system 32 through the filter and passed directly into the blower 34. The filtered air is then passed into the inner environment 28 from the blower. Accordingly, air from the outer environment 30 is passed into the inner environment 28 through the filter 36. FIG. 5 shows an alternative example of a filter 36 and blower 34 in fluid communication with each other. Filter 36 and blower 34 are spaced apart from each other. The blower 34 receives air from the inner environment 28 and exhausts the air into the outer environment 30. Consequently, air from the outer environment 30 is drawn into the filter 36 and filtered air is passed into the inner environment.

FIGS. 1-5 show the blower 34 isolated from the outer environment 30 where the blower 34 is disposed within the inner environment 28. FIG. 6 shows the blower 34 isolated from the outer environment 30 and not disposed within the inner environment 28. Rather, blower 34 is sectioned off with partition 44 from the inner environment 28. The blower 34 need not be hermetically sealed from both the inner and outer environments, but may set off by a pouch or pocket in the barrier 22. Filtered air can be delivered from the blower 34 to the inner environment 28 with a conduit 46. In the example of a chemical suit, the blower may be disposed within the chemical suit and filtered air is delivered to a hood via the conduit. The hood may be sectioned off from the chemical suit. In such a case, the environment within the hood may be considered to be the inner environment 28.

The blower is driven by a power source that can include a battery, pneumatically or connected to an external power source. If the power source is located externally, the barrier may need to be fitter with fluid-tight pass-throughs to efficiently provide power to the blower.

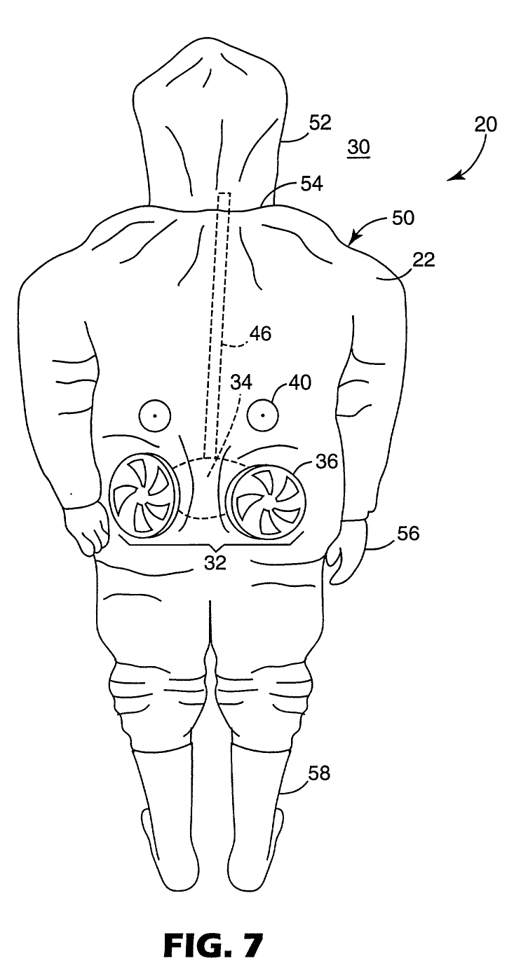

FIG. 7 shows a perspective view of a user within a personal containment device 20 in the form of a chemical suit 50. The suit includes an integrally formed hood 52. The hood is drawn at the neck with an elastic band, or neck band 54. The cuffs of the legs and arms of the chemical suit 50 also include elastic bands (not shown). The personal containment device 20 is sealed from the outer environment 30 with attachments such as gloves 56 and boots 58. The suit 50 includes air delivery system 32 including a blower 34 (shown in phantom) and filters 36 located near the lower back of the user. The air delivery system 32 can be secured in place in a number of ways. For example, the blower 34 can be placed in a pouch or pocket within the suit 50, can be worn on a belt around the waist of a user, or the like. Vents 40 are included in the suit 50 above the filters 36. A conduit 46 (shown in phantom) is attached to the blower 34 and extends up the back of the suit 50, through the elastic neck band 54 and is terminated within the hood 52. Air from the outer environment 30 is drawn into the filters 36 interfacing with the outer environment 30 and filtered air is delivered to the inner environment 28 via the blower 34. Air is expelled into the outer environment 30 through vents 40 once the pressure with the suit 50 has exceeded a threshold.

The chemical suit 50 is constructed from readily available materials and parts. The blower 34 and filter 36 are available from 3M Company, St. Paul, Minn., U.S.A. and is sold in the United Kingdom under the trade designation Jupiter. The blower 34 runs on a battery (not shown). For example, the battery may come in four different varieties: a four hour battery, an eight hour battery, an intrinsically safe battery, and a lithium battery. The lithium battery is particularly suited for applications where the suit may sit on a shelf for several years before it is needed. The lithium battery is currently not rechargeable, whereas the first three batteries are rechargeable. The filter can be suited for the particular application or contaminant. One suitable filter is available from the 3M Company and sold as an ABEK P3 filter for general purpose use. The filter is suited for outer environments where contaminants may include chemical and biological weapons. The suit is available from Respirex of Redhill Surrey, England, and is constructed from a high performance chemical barrier available from DuPont and sold under the trade designation of Tychem TK. Other materials are contemplated. Another example of a barrier is a medium to low chemical barrier available from DuPont sold under the trade designation Tyvek F. Barriers may be selected based on the intended application. Also, a suit 50 may include a combination of barriers such as a body portion constructed from a heavier high performance chemical barrier and the hood may be constructed from a lighter medium to low performance chemical barrier.

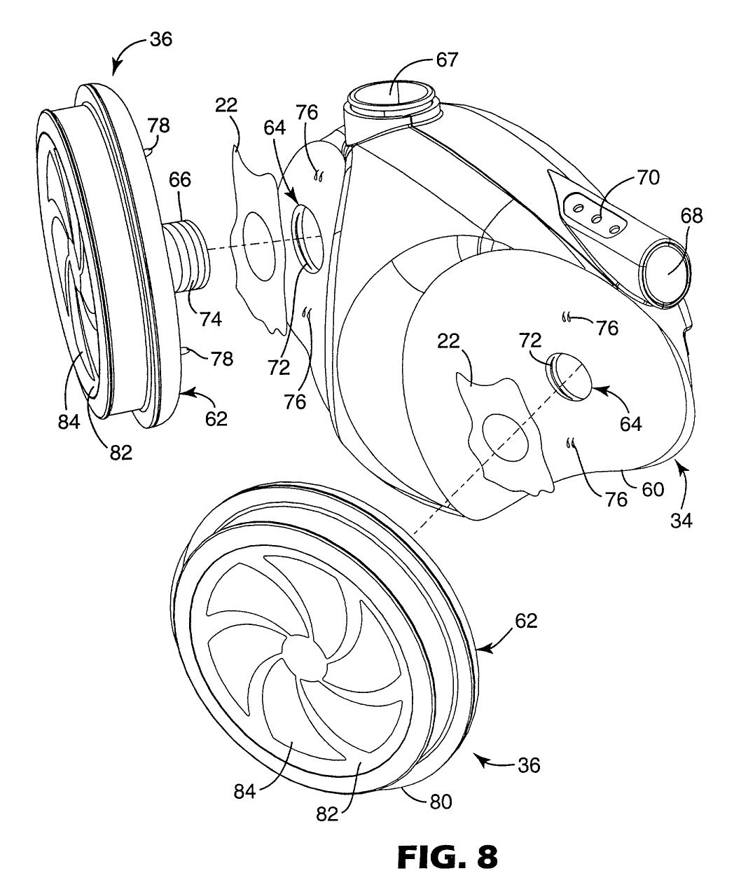

FIG. 8 shows an exploded perspective view of the air delivery system 32 including a blower 34 and a filter 36. Specifically in the example, the air delivery system 32 includes a blower housing 60, one or more replaceable filter cartridges 62, housing fluid inlets 64 and a filter fluid outlet 66, and housing fluid outlet 67. A motor is provided within the housing 60 to draw fluid from the housing fluid inlet 64 and pass it under pressure through the housing fluid outlet 67. The housing fluid outlet mates with the conduit 42 to provide fluid to the inner environment. The housing fluid outlet 66 may be provided with a swivel to reduce strain on the conduit 46. The housing also includes a power switch 68 and an indicator 70 providing a variety of alerts such as whether air flow has dropped below a certain threshold.

Each filter cartridge 62 includes a filter housing 80 having a major surface 82. Filter media 84 is retained within an internal chamber defined by the filter housing 80. Openings in the major surface 82 permit fluid to be drawn into the filter media 84 and is passed through the filter media 84 and out the filter fluid outlet 66. The filter cartridge 62 is sealably mounted to the blower housing 60 at the filter fluid outlet 66 and the housing fluid inlet 64.

The housing fluid inlets 64 have female threads 72 which are adapted to mate with male threads 74 on the filter fluid outlet 66. Each of the threads 72 is highly pitched and extends only about once around the inner circumference of the fluid inlet 64. Housing detents 76 are spaced radially around the fluid inlet 64 and align with filter detents 78 on filter cartridges 62 when the filter cartridge 62 and housing 60 are engaged. Housing detents 76 engage and releasably lock filter detents 78 when the filter cartridge 62 is sealably mounted on the housing 60. The housing 60 can also include a deformable inlet gasket (not shown) located within the housing inlet 64.

FIG. 9 shows an example of an interface 42 introduced in FIG. 4. The interface 42 of the example includes two parts, an inner adapter 86 and an outer adapter 88. The interface 42 provides a mechanical seal around a hole in the barrier 22 for the air-delivery system 32 that does not need an adhesive to seal the barrier 22. The inner adapter 86 includes a protruding member 90 that is sized to mate with the housing fluid inlet 64. The protruding member 90 includes a male thread 92 that, in one example, extends about twice around the protruding member 90. The inner adapter 86 also includes a tongue 94 that extends axially from a flange 96. The tongue 94 includes a female thread 98 that extends around the inner circumference of the tongue 94. The tongue 94 mates with an annular channel (not shown) on the outer adapter 88. The annular channel includes a male thread adapted to mate with the female thread 98 of the tongue 94. The annular channel is disposed within boss 100 protruding from the outer adapter 88. A thread 102 extends around the inner circumference of the boss 100, and the thread 102 is designed to mate with the male threads 74 on the filter fluid outlet 66 of the filter cartridge 62. Also, the flange 96 of the inner adaptor 86 includes an annular groove 104 that is designed to mate with a tongue (not shown) on the flange 106 of the outer adapter 88. The adaptors 86, 88 mate together to create a fluid-tight seal with the barrier 22, which forms a gasket between the adaptors 86, 88. A plurality of adapter detents 108 are disposed on the boss 100 to mate with detents on the filter cartridge 62 and releasably lock the filter cartridge to the interface 42. Gaskets may also be used to seal the filter cartridge, interface and blower housing. In the example, the interface 42 is formed of a thermoplastic that is chemically inert and mechanically strong enough to hold a thread. One suitable material is a glass filled polypropylene. The amount of glass filler maybe 10 percent to 30 percent by weight.

Although the personal containment system and its components have been described with reference to examples, or embodiments, it is to be understood that changes may be made in form and detail without departing from the spirit and scope of the invention.

We claim:

1. A personal containment system, comprising:
a generally fluid-tight barrier having an inner surface and an outer surface, the generally fluid tight barrier adapted to at least partially define an inner environment interfacing with the inner surface, and an outer environment interfacing with the outer surface; and
an air delivery system connected through the generally fluid-tight barrier, the air delivery system comprising:
a blower generally isolated from the outer environment; and
a filter in fluid communication with the blower and interfacing with the outer environment, wherein air from the outer environment is passed into the inner environment through the filter.

2. The personal containment system of claim 1 wherein the generally fluid-tight barrier defines the inner environment and the outer environment.

3. The personal containment system of claim 1 wherein the generally fluid-tight barrier includes a vent adapted to allow gases to pass from the inner environment to the outer environment and to prevent fluid to pass from the outer environment to the inner environment.

4. The personal containment system of claim 1 wherein the blower includes a battery.

5. The personal containment system of claim 1 wherein the blower is disposed in the inner environment.

6. The personal containment system of claim 1 wherein the filter is detachable from the blower.

7. The personal containment system of claim 1 wherein the filter is substantially disposed within the outer environment.
